(19) United States
(12) Patent Application Publication
(10) Pub. No.: US 2008/0226568 A1
(43) Pub. Date: Sep. 18, 2008

(54) Synergistic preservative systems and their use in topical compositions

(75) Inventors: Ellen Rozsa, Bergenfield, NJ (US); Grace Riccardi, Jersey City, NJ (US); Ben Ramirez, Englewood, NJ (US)

Correspondence Address: Charles N.J. Ruggiero;Ohlandt , Greeley, Ruggiero & Perle, L.L.P., 10th Floor, One Landmark Square, Stamford, CT, 06901-2682, US

(73) Assignee: Playtex Products, Inc.

(21) Appl. No.: 11/717,390

(22) Filed: Mar. 13, 2007

(51) Int. Cl. A61K 38/43; A61K 38/54; A61P 31/04; A61P 31/10

(52) U.S. Cl. 424/59; 424/94.1; 424/94.2

(57) ABSTRACT

A synergistic preservative system having an oil-miscible glycol and an enzymatic composition. The synergistic preservative system demonstrates enhanced anti-fungal, anti-bacterial, and anti-microbial efficacy and is free of formaldehyde donors, parabens, ethanols, and isothiazolinones.

### BACKGROUND OF THE INVENTION

1. Field of the Invention

The present disclosure relates generally to topical compositions. More particularly, the present disclosure relates to synergistic preservative systems and their use in topical compositions.

2. Description of Related Art

Preservatives are frequently used in all types of topical compositions such as, but not limited to, cosmetic compositions, dermatological compositions and others, to prevent decay and breakdown of such compositions mainly caused by microorganisms. Effective preservatives must have two types of activity, namely anti-microbial activity and anti-fungal activity. Most of the current preservatives contain some form of paraben, often in combination with phenols and quaternary compounds. Some of these paraben preservative systems, however, are limited in their global acceptability and are not allowed in either Europe or Japan. Various parabens have shown estrogenic activity and other deleterious effects on reproductive systems in various test models.

Likewise, while effective, other preservative systems, such as, formaldehyde donors, isothiazolinones, and ethanols fail to meet numerous safety and compatibility-related issues, for example, high irritation potential and incompatibility with avobenzone.

It is known that certain glycols offer some anti-microbial efficacy at relatively high concentrations, for example, in the range between 1% to 7% based on a total weight of a composition. However, glycols have limited or no anti-fungal efficacy. Therefore, glycol, by itself, cannot substitute the anti-bacterial and anti-fungal properties of a preservative in a typical topical composition.

Accordingly, while there have been attempts in the prior art to formulate paraben-free compositions, there is still a strong need in the art for a preservative composition or system that is paraben-free, has less irritation potential, has both antimicrobial and fungicidal activity, and has lower manufacturing costs. The present disclosure meets these needs.

### BRIEF SUMMARY OF THE INVENTION

The present disclosure provides a synergistic preservative system having an oil-miscible glycol and an enzymatic composition. The synergistic preservative system demonstrates enhanced anti-fungal and/or anti-bacterial efficacy and is free of a formaldehyde donor, paraben, ethanol, and isothiazolinone.

In some embodiments, the synergistic preservative system has an oil-miscible glycol.

In an embodiment, the oil-miscible glycol in the synergistic preservative system is caprylyl glycol.

In some embodiments, the synergistic preservative system has an enzymatic composition that has glucose, glucose oxidase and lactoperoxidase.

In some embodiments, the synergistic preservative system will also have one or more preservative components selected from the group consisting of essential oils, essential oil derivatives, plant extracts, plant extract derivatives, plant extract botanical derivatives, vegetable oils, vegetable oil derivatives, fatty acids, fatty acid derivatives, or any combinations thereof.

A topical composition will have a synergistic preservative system. The synergistic preservative system has an oil-miscible glycol and an enzymatic composition. Additionally, the topical composition is free of formaldehyde donor, paraben, ethanol, and isothiazolinone.

In additional embodiments, the topical composition can be in the form of a lotion, cream, spray, gel, foam, powder, stick, or any combination thereof.

In other embodiments, the topical composition may have one or more additional ingredients selected from the group consisting of a solvent, a surfactant, a emulsifier, an emollient, a humectant, a moisturizer, a thickener, a botanical, an antioxidant, a vitamin, a sunscreen agent, a preservative, a pH adjuster, a chelating agent, a viscosity modifier, or any combinations thereof.

The above-described and other advantages, features, and benefits of the present disclosure will be appreciated and understood by those skilled in the art from the following detailed description, drawings, and appended claims.

### DETAILED DESCRIPTION OF THE INVENTION

The present disclosure provides enhanced or synergistic preservative systems for use in topical compositions. Such topical compositions include, for example, cosmetic and dermatological compositions such as, but not limited to, suncare and screen products. In particular, it has been found that a preservative system with an oil-miscible glycol, an enzymatic composition, and, optionally, one or more preservative components, results in a synergistic preservative system that is effective for use in topical compositions.

These synergistic preservative systems demonstrate adequate anti-microbial and anti-fungal efficacy without the use of harsher preservatives, such as, formaldehyde donor, paraben, ethanol, and isothiazolinone. In one embodiment of the present disclosure, the synergistic preservative system is free of formaldehyde donors, parabens, ethanols, and isothiazolinones. The synergistic preservative systems of the present disclosure may support preservative-free product claims and may also provide anti-oxidant and moisturization benefits.

When one or more oil-miscible glycols, the enzymatic composition, and optionally, one or more preservative components are combined according to the present disclosure, the combination unexpectedly results in a synergistic preservative system that is free of harsh preservatives, but still has anti-fungal, anti-bacterial, and anti-microbial efficacy. Furthermore, as a result of the synergism between the one or more oil-miscible glycols, the enzymatic composition, and the optional one or more preservative components, a reduced concentration of each component is required to achieve anti-fungal, anti-bacterial, and/or anti-microbial efficacy in topical compositions.

Preferably, a suitable oil-miscible glycol for use in the present disclosure is caprylyl glycol which is, by way of example, sold by Inolex Chemical Company under the tradename Lexgard O.

The oil-miscible glycol is present in the final topical composition in an amount about 0.1 wt. % to about 5 wt. %, based on the total weight of the composition. Preferably, the oil-miscible glycol is present in an amount of about 0.25 wt. % to about 5 wt. %, and most preferably in an amount of about 1.25 wt. % to about 2 wt. %, and any subranges therebetween, based on the total weight of the composition.

Suitable enzymatic compositions for use in the present disclosure will exhibit sufficient metabolic processes to inhibit and eventually kill harmful microbes that invade the topical composition resulting in degradation. It is foreseen that suitable enzymatic compositions include at least one enzyme and a substrate.

In one embodiment of the present disclosure, the enzymatic composition is a combination of glucose, glucose oxidase, and lactoperoxidase. This enzymatic composition is present in the final topical composition in an amount about 1.05% wt. based on the total weight of the composition.

In the enzymatic composition, glucose is present in an amount about 1.0 % wt. based on the total weight of the topical composition. In the enzymatic composition, the sum total of the combination of glucose oxidase and lactose peroxidase is present in an amount of about 0.5 wt. % based on the total weight of the topical composition.

Additionally, the glucose oxidase is present in the enzymatic composition in an amount about 1500 units/ml to about 2750 units/ml, based on the total weight of the sum total of the combination of the glucose oxidase and lactose peroxidase present in the enzymatic composition.

Furthermore, the lactoperoxidase is present in the enzymatic composition in an amount about 1000 units/ml to about 1800 units/ml based on the total weight of the sum total of the combination of the glucose oxidase and lactose peroxidase present in the enzymatic composition.

In an exemplary embodiment, the enzymatic composition is sold by Arch Personal Care Products, L.P. under the tradename “Biovert Enzyme & Substrate”.

Without wishing to be bound by any particular theory, it is believed that the glucose oxidase, in the presence of oxygen, reacts with the glucose to generate hydrogen peroxide. The hydrogen peroxide is used by the lactoperoxidase to catalyze the oxidation of SCN and I anions forming hypoiodite and hypothiocyanate. The hypoiodite and hypothiocyanate demonstrate anti-microbial activity that results in the rapid death of the microbial cell. However, the enzymatic composition alone does not provide adequate preservation properties and can cause instability of the pH. It is believed, however, that the combination of Biovert and the caprylyl glycol will enhance the antifungal efficacy. Consequently, the combination will yield effective synergystic preservative efficacy.

It is also contemplated that the synergistic preservative system according to the present disclosure can include one or more preservative components that can enhance anti-microbial efficacy. Additionally, it is foreseen that the preservative components can impart bioactivity and functionality of the synergistic preservative system.

Suitable preservative components do not include formaldehyde donors, parabens, ethanols, and isothiazolinones. Rather, the present disclosure contemplates that suitable preservative components include, but are not limited to, an essential oil, essential oil derivative, plant extract, plant extract derivative, plant extract botanical derivative, vegetable oil, vegetable oil derivative, fatty acid, fatty acid derivative, or any combinations thereof.

In one embodiment of the present disclosure, the preservative component is willow bark powder.

Suitable essential oils include, but are not limited to, tea tree oil. Without wishing to be bound by any particular theory, it is believed that the tea tree oil combined with the caprylyl glycol and the enzymatic composition will reduce the potential of irritation as well as prevent an interference with the pH, color, viscosity, and odor of the topical composition formulations. In a preferred embodiment of the present disclosure, the tea tree oil, by way of example, is commercially available from Active Concepts/Southern Cross Botanicals under the trade name Melafresh T96 Tea Tree Oil; Melafresh T10-WS-Water Soluble Tea Tree Oil.

In a preferred embodiment, the tea tree oil is present in the final cosmetic composition in an amount about 0.1 wt. % to about 5.0 wt. %, based on the total weight of the composition, preferably in an amount about 0.1 wt. % to about 3.0 wt. %, and most preferably in an amount about 0.5 wt. % to about 1.0 wt. %, and any subranges there between.

It is also contemplated that the white willow bark extract powder is present in the final cosmetic composition in an amount about 0.1 wt. % to about 3.0 wt. %, based on the total weight of the composition, preferably in an amount about 0.1 wt. % to about 1.0 wt. %, and most preferably in an amount about 0.5 wt. % to about 1.0 wt. %, and any subranges there between.

In an additional embodiment, it is contemplated that the synergistic preservative system may include one or more additional ingredient including, but not limited to, solvents, surfactants, emulsifiers, emollients, humectants, moisturizers, thickeners, antioxidants, vitamins, sunscreen agents, additional preservatives, pH adjusters, chelating agents, viscosity modifiers, or any combinations thereof.

In another embodiment, a topical composition is formulated including the synergistic preservative system discussed above. It is contemplated that the topical composition may be in any known composition. For example, the topical composition may be, but is not limited to, a lotion, cream, spray, gel, foam, powder, stick, or any combinations thereof. It is also contemplated that the topical composition can be delivered utilizing both woven and non-woven substrates.

Additionally, it is contemplated that the topical composition may include one or more additional ingredients including, but not limited to, solvents, surfactants, emulsifiers, emollients, humectants, moisturizers, thickeners, botanicals, antioxidants, vitamins, sunscreen agents, additional preservatives, pH adjusters, chelating agents, viscosity modifiers, or any combinations thereof.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

1. A synergistic preservative system for use in a topical composition, comprising:
an oil-miscible glycol; and
an enzymatic composition,
wherein the synergistic preservative system demonstrates enhanced anti-fungal and/or anti-bacterial efficacy and is free of formaldehyde donors, parabens, ethanols, and isothiazolinones.

2. The synergistic preservative system of claim 1, wherein said oil-miscible glycol is caprylyl glycol.

3. The synergistic preservative system of claim 1, wherein said oil-miscible glycol is present in an amount between 0.1 wt. % and 5 wt. % based on the total weight of the topical composition.

4. The synergistic preservative system of claim 1, wherein said oil-miscible glycol is present in an amount between 0.25 wt. % and 5 wt. % based on the total weight of the topical composition.

5. The synergistic preservative system of claim 1, wherein said oil-miscible glycol is present in an amount between 1.25 wt. % and 2 wt. % based on the total weight of the topical composition.

6. The synergistic preservative system of claim 1, wherein said enzymatic composition comprises glucose, glucose oxidase and lactoperoxidase.

7. The synergistic preservative system of claim 1, wherein said enzymatic composition is present in said synergistic system in an amount about 1.05 wt. % based on the total weight of the topical composition.

8. The synergistic preservative system of claim 6, wherein said glucose is present in said enzymatic composition in an amount about 1.0 wt. % based on the total weight of the topical composition.

9. The synergistic preservative system of claim 6, wherein said glucose oxidase is present in said enzymatic composition in an amount between 1500 units/ml and 2750 units/ml, based on the total weight of the sum total of the glucose oxidase and lactoperoxidase present in the enzymatic composition.

10. The synergistic preservative system of claim 6, wherein said lactoperoxidase is present in said enzymatic composition in an amount between 1000 units/ml and 1800 units/ml, based on the total weight of the sum total of the combination of the glucose oxidase and lactose peroxidase present in the enzymatic composition.

11. The synergistic preservative system of claim 1, further comprising one or more preservative components.

12. The synergistic preservative system of claim 11, wherein said one or more preservative components is selected from the group consisting of essential oils, essential oil derivatives, plant extracts, plant extract derivatives, plant extract botanical derivatives, vegetable oils, vegetable oil derivatives, fatty acids, fatty acid derivatives, or any combinations thereof.

13. The synergistic preservative system of claim 12, wherein said plant extract botanical derivative comprises willow bark powder.

14. A topical composition comprising:
a synergistic preservative system comprising an oil-miscible glycol and an enzymatic composition,
wherein the topical composition is free of formaldehyde donors, parabens, ethanols, and isothiazolinones.

15. The topical composition of claim 14, wherein said oil-miscible glycol is caprylyl glycol.

16. The topical composition of claim 14, wherein said oil-miscible glycol is present in said topical composition in an amount between 0.1 wt. % and 5 wt. % based on the total weight of the topical composition.

17. The topical composition of claim 14, wherein said enzymatic composition is present in said topical composition in an amount about 1.05 wt. % based on the total weight of the topical composition.

18. The topical composition of claim 14, wherein said enzymatic composition comprises glucose, glucose oxidase, and lactoperoxidase.

19. The topical composition of claim 14, wherein the topical composition is in a form selected from the group consisting of a lotion, cream, spray, gel, foam, powder, stick, or any combinations thereof.

20. The topical composition of claim 14, further comprising one or more additional ingredients selected from the group consisting of a solvent, a surfactant, a emulsifier, an emollient, a humectant, a moisturizer, a thickener, a botanical, an antioxidant, a vitamin, a sunscreen agent, a preservative, a pH adjuster, a chelating agent, a viscosity modifier, or any combinations thereof.

21. The topical composition of claim 20, wherein said botanical comprises willow bark powder.
